(19) United States
(12) Patent Application Publication
(10) Pub. No.: US 2024/0139083 A1
(43) Pub. Date: May 2, 2024

(54) BLEACHING COMPOSITION FOR KERATIN FIBERS

(71) Applicant: KAO GERMANY GMBH, Darmstadt (DE)

(72) Inventors: Steven BREAKSPEAR, Darmstadt (DE); Peter BAUER, Darmstadt (DE); Bernd NÖCKER, Darmstadt (DE); Ines RABELO DE MORAES, Darmstadt (DE)

(73) Assignee: KAO GERMANY GMBH, Darmstadt (DE)

(21) Appl. No.: 18/275,085

(22) PCT Filed: Feb. 25, 2022
(86) PCT No.: PCT/EP2022/054764

§ 371 Date: Jul. 31, 2023

(30) Foreign Application Priority Data
Feb. 26, 2021 (EP) 21159448.6

(51) Int. Cl. A61K 8/22; A61K 8/23; A61K 8/25; A61K 8/34; A61K 8/41; A61K 8/60; A61Q 5/06; A61Q 5/08

(52) U.S. Cl. CPC A61K 8/22; A61K 8/23; A61K 8/25; A61K 8/345; A61K 8/41; A61K 8/60; A61Q 5/065; A61Q 5/08; A61K 2800/4322; A61K 2800/88

(57) ABSTRACT

Bleaching compositions for keratin fibers are disclosed and comprise a) one or more persalt(s) and/or peroxy salt(s), b) one or more alkalizing agent(s), c) one or more non-acetylated sugar alcohol(s), and/or their mixtures. Total concentrations of compound(s) according to group c) is in the range of 0.0001% to 2.5% by weight, calculated to total weights of the bleaching compositions, the bleaching compositions comprise less than 5% by weight of water, calculated to the total weights of the bleaching compositions, and one or more compound(s) according to group c) is/are mannitol, xylitol, and/or their mixtures.

### FIELD OF THE INVENTION

The invention is directed to the provision of a bleaching composition for keratin fibers, a kit-of-parts for bleaching, and a method for bleaching keratin fibers.

### BACKGROUND OF THE INVENTION

Many consumers are unsatisfied with their hair color. Changes of hair color, in most cases, require the lightening of hair, especially for dark hair customers. A common technique is hair bleaching of whole hair as well partial treatments such as streaks. For such treatments a high degree of lightening is desired.

One way to increase lightening performance of bleaching compositions is the use of a reductive pre-treatment step, e.g. by using organic thiols (EP 2 608 848 B1) or inorganic reducing agents such as sulfites. However, with a reductive pre-treatment step hair lightening and hair damage are increased simultaneously.

EP0525239 discloses the use of acetylated sugar alcohols at concentrations of 45 by weight or more to increase the performance of perborate bleach for textile fibers.

EP3040065 discloses the use of mono- and disaccharides in aqueous oxidizing composition.

Thus, there is a real need to develop bleaching composition, which deliver an increased degree of lightening without leading to high degrees of damage.

### SUMMARY OF THE INVENTION

Therefore, the first object of the present invention is a bleaching composition A for keratin fibers, preferably for human keratin fibers, more preferably for human hair, comprising
a) one or more persalt(s) and/or peroxy salt(s),
b) one or more alkalizing agent(s),
c) one or more non-acetylated sugar alcohol(s), and/or their mixtures,
wherein the total concentration of compound(s) according to group c) is in the range of 0.0001% to 2.5% by weight, calculated to the total weight of the bleaching composition A,
wherein the bleaching composition A comprises less than 5% by weight of water, calculated to the total weight of the bleaching composition A, and wherein one or more compound(s) according to group c) is/are mannitol, xylitol, and/or their mixtures.

The second object of the present invention is a two-part bleaching composition for keratin fibers, preferably for human keratin fibers, more preferably for human hair, comprising a bleaching composition A as defined above and a second aqueous oxidizing composition B, preferably comprising hydrogen peroxide and having a pH in the range of 1 to 6.

The third object of the present invention is a kit-of-parts for bleaching and dyeing keratin fibers, preferably human keratin fibers, more preferably human hair comprising:
a bleaching composition A as defined above,
a second aqueous oxidizing composition B, preferably comprising hydrogen peroxide and having a pH in the range of 1 to 6,
a third composition comprising one or more dye(s), preferably one or more oxidative hair dye(s) and/or one or more hair direct dye(s), and/or their mixtures.

The fourth object of the present invention is a method for bleaching keratin fibers, preferably human keratin fibers, more preferably human hair, comprising the steps of:
i) mixing a bleaching composition A as defined above with a second oxidizing composition B, preferably comprising hydrogen peroxide and having a pH in the range of 1 to 6, to yield a ready-to-use composition having a pH in the range of 7 to 12,
ii) applying the ready-to-use composition onto keratin fibers and leaving it for a time period of 1 min to 60 min,
iii) rinsing-off the keratin fibers with water and optionally shampooing the keratin fibers.

A fifth object of the present invention is a method for dyeing of keratin fibers, preferably human keratin fibers, more preferably human hair, comprising the steps of:
iv) providing the bleaching composition A as defined above and mixing it with an aqueous oxidizing composition B, preferably comprising hydrogen peroxide and having a pH in the range of 1 to 6, to yield a ready-to-use composition having a pH in the range of 7 to 12,
v) applying the ready-to-use composition onto keratin fibers and leaving it for a time period of 1 min to 60 min,
vi) rinsing-off the keratin fibers with water and optionally shampooing the keratin fibers,
vii) providing a dyeing composition comprising one or more dye(s) selected from oxidative dye coupler, oxidative dye precursor, and direct dye, and/or their mixtures, and comprising one or more alkalizing agent(s),
viii) mixing the composition of step vii) with an aqueous oxidative composition B to form a ready-to-use dyeing composition having a pH in the range of 7 to 12,
ix) applying the ready-to-use dyeing composition onto keratin fibers and leaving it for a time period of 1 min to 60 min,
x) repeating step vi).

### DETAILED DESCRIPTION OF THE INVENTION

Inventors of the present invention have unexpectedly found out that the objects of the present invention increase bleaching performance while not leading to elevated damage of keratin fibers. Moreover, due to the mildness of the compositions to fibers, they feel more cosmetic, healthier, and appear to have more shine.

### Bleaching Composition A

The present invention is directed to a bleaching composition A for keratin fibers, preferably for human keratin fibers, more preferably for human hair, comprising
a) one or more persalt(s) and/or peroxy salt(s),
b) one or more alkalizing agent(s),
c) one or more non-acetylated sugar alcohol(s), and/or their mixtures,
wherein the total concentration of compound(s) according to group c) is in the range of 0.0001% to 2.5% by weight, calculated to the total weight of the bleaching composition A,
wherein the bleaching composition A comprises less than 5% by weight of water, calculated to the total weight of the bleaching composition A,
and wherein one or more compound(s) according to group c) is/are mannitol, xylitol, and/or their mixtures.

Preferably, the bleaching composition A comprises less than 1% by weight of water, more preferably it is an anhydrous composition, from the viewpoint of stability. The term anhydrous is to be understood that no water is added to the powder. However, this does not exclude any water bound to the ingredients by, for example, capillary forces.

### Compound(s) According to Group a)

The bleaching composition A comprises one or more persalt(s) and/or peroxy salt(s) as compound(s) according to group a). Suitable persalts and/or peroxy salts are sodium persulfate, potassium persulfate, ammonium persulfate, earth alkali peroxides such as magnesium peroxide, melamine peroxide or urea peroxide or phthalimidoperoxy hexanoic acid. The preferred persalts from the viewpoint of bleaching power are sodium, potassium, and ammonium persulfate.

It is preferred from the viewpoint of bleaching power and cosmetic safety that the total concentration of persalts and/or peroxy salts in the bleaching composition A is in the range of 10% to 80% by weight, preferably 15% to 70% by weight, more preferably 20% to 60% by weight, and still more preferably 25% to 60% by weight, calculated to the total weight of the bleaching composition A.

### Compound(s) According to Group b)

The bleaching composition A further comprises one or more alkalizing agent(s) as compounds according to group b). Preferably, the compounds according to group b) are selected from inorganic and/or organic alkalizing agent(s), and/or their mixtures.

It is preferred from the viewpoint of stability and bleaching power that the compounds according to group b) are inorganic alkalizing agent(s), preferably selected from metasilicates, carbonates, and/or bicarbonates, and/or their alkali or earth alkali salts, and/or their mixtures, more preferably the compound according to group b) is sodium metasilicate.

It is preferred from the viewpoint of alkalinity that the compounds according to group b) are organic alkalizing agent(s), preferably selected from alkyl- or alkanolamines according to the general structure

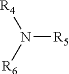

wherein R₄, R₅, and R₆ are same or different H, from C₁ to C₄, C₃ to C₄ unsaturated alkyl, C₃ to C₄ branched alkyl, C₁ to C₄ hydroxyl alkyl, C₃ to C₄ unsaturated hydroxyl alkyl, C₃ to C₄ branched hydroxyl alkyl, with the condition that at least one of R₄, R₅, or R₆ is different from H, and/or their mixtures.

Suitable organic alkalizing agents are monoethanolamine, diethanolamine, triethanolamine, monomethylamine, dimethylamine, trimethylamine, monoethylamine, diethylamine, and 2-aminomethyl propanol.

The most preferred organic alkalizing agent(s) as compounds according to group b) are selected from monoethanolamine and/or 2-aminomethyl propanol.

It is preferred from the viewpoint of alkalinity and stability that the composition comprises one or more compound according to group b) at a total concentration in the range of 0.25% to 30% by weight, preferably 0.5% to 25% by weight, more preferably 1% to 20% by weight, calculated to the total weight of the bleaching composition A.

Optionally, the bleaching composition A may comprise one or more ammonium salt(s) different from persalt(s) and peroxy salt(s).

Suitable ammonium salts different from persalt(s) and peroxy salt(s) are ammonium carbonate, ammonium hydrogen carbonate, ammonium carbamate, ammonium chloride, ammonium sulfate, ammonium phosphates, ammonium nitrate, ammonium bromide, ammonium iodide, ammonium thiosulfate, ammonium molybdate, ammonium vanadate, ammonium sulfamate, ammonium citrate, ammonium salicylate, ammonium valerate, ammonium tartarate, ammonium benzoate, ammonium acetate, ammonium formiate and ammonium lactate. The bleaching composition A may also comprise mixtures of ammonium salts.

The bleaching composition A may comprise one or more ammonium salts different from persalt(s) and peroxy salt(s) at a total concentration in the range of 0.1% to 10% by weight, calculated to the total weight of the composition.

### Compound(s) According to Group c)

The bleaching composition A of the present invention comprises one or more non-acetylated sugar alcohol(s), and/or their mixtures, as compound(s) according to group c), wherein the total concentration of compound(s) according to group c) is in the range of 0.0001% to 2.5% by weight, calculated to the total weight of the bleaching composition A.

It is further preferred from the viewpoint of bleaching power that the one or more non-acetylated sugar alcohol(s) as compound(s) according to group c) is/are mannitol, xylitol, and/or their mixtures, preferably it is mannitol.

It is further preferred from the viewpoint of bleaching power that the total concentration of compound(s) according to group c) is in the range of 0.0005% to 0.5% by weight, preferably in the range of 0.001% to 0.25% by weight, more preferably in the range of 0.001% to 0.1% by weight, calculated to the total weight of the bleaching composition A.

The disclosure of the present invention also includes a bleaching composition A for keratin fibers, preferably for human keratin fibers, more preferably for human hair, comprising
a) one or more persalt(s) and/or peroxy salt(s),
b) one or more alkalizing agent(s),
c) one or more non-acetylated sugar alcohol(s), and/or their mixtures,
wherein the total concentration of compound(s) according to group c) is in the range of 0.0001% to 2.5% by weight, preferably in the range of 0.0001% to 1% by weight, more preferably in the range of 0.0001% to 0.1% by weight, calculated to the total weight of the bleaching composition A, and
wherein the bleaching composition A comprises less than 1% by weight of water, calculated to the total weight of the bleaching composition A, preferably it is anhydrous.

### Cosmetic Forms of Bleaching Composition A

The bleaching composition A of the present invention may be in the form of a bleaching powder composition.

For preparation of the bleaching powder composition, an excipient may be added. Such an excipient is diatomaceous earth.

It is further preferred from the viewpoint of cosmetic safety that the bleaching powder composition is dust-free. This property can commonly be achieved by adding lipophilic compounds to the bleaching powder. From this viewpoint, the composition comprises one or more lipophilic compound(s) as compound according to group d).

The bleaching composition A may be in the form of a bleaching paste composition. It is further preferred that the bleaching paste comprises one or more lipophilic compound(s) as compound according to group d).

Preferably, the compound according to group d) is selected from C₁₂ to C₂₂ fatty alcohols, esters of C₃ to C₁₂ alcohols with C₁₂ to C₂₂ fatty acids, C₈ to C₂₂ fatty acids, vegetable oils, and/or silicones, and/or hydrocarbon-based products, and/or their mixtures.

Suitable C₁₂ to C₂₂ fatty alcohols are myristyl alcohol, cetyl alcohol, stearyl alcohol, behenyl alcohol, and cetearyl alcohol.

Suitable esters of C₃ to C₂₂ alcohols with C₁₂ to C₂₂ fatty acids are isopropyl myristate, isopropyl palmitate, and myristyl myristate.

Suitable C₈ to C₂₂ fatty acids are oleic acid, linoleic acid, and palmitic acid.

Suitable vegetable oils are olive oil, almond oil, sunflower oil, and argan oil.

Suitable silicones are non-aminated and/or aminated silicones. The latter are commonly known as amodimethicones.

Suitable hydrocarbon-based products are mineral oil, paraffins, and Vaseline.

It is preferred from the viewpoint of making the bleaching composition A dust-free or formulating it as a paste that the concentration of compounds according to group d) is in the range of 1% to 20% by weight, preferably 2% to 15% by weight, more preferably 3% to 12% by weight, calculated to the total weight of the bleaching composition A.

### Optional Compounds—Surfactants as Compounds According to Group e)

The bleaching composition A of the present invention may further comprise one or more surfactant(s) as compound according to group e), preferably selected from non-ionic surfactants, anionic surfactants, cationic surfactants, and/or amphoteric/zwitterionic surfactants, and/or their mixtures, more preferably selected from anionic surfactants and/or non-ionic surfactants, from the viewpoint of stabilizing the composition and improving wettability and mixability.

Preferably, the anionic surfactants may be selected from ethoxylated or non-ethoxylated alkyl ether sulfate surfactants, alkyl sulfates, ethoxylated and/or non-ethoxylated alkyl carboxylates, ethoxylated or non-ethoxylated amino acid surfactants, and/or their mixtures.

Suitable examples are alkyl sulfate or preferably ethoxylated alkyl ether sulfate surfactant or mixtures thereof having an alkyl chain length of C₁₀ to C₂₂.

Suitable non-ionic surfactants may be selected from alkyl polyglycosides, ethoxylated triglycerides, ethoxylated fatty alcohols, ethoxylated fatty acid esters, and/or their mixtures.

Suitable cationic surfactants are quaternary ammonium surfactants having a carbon chain length in the range of C₁₂ to C₂₂ or surfactants having a tertiary amine group and at least one alkyl chain having a carbon chain length in the range of C₁₂ to C₂₂ such as alkylamidoalkylamine surfactants. Suitable examples are cetrimonium chloride.

Suitable amphoteric/zwitterionic surfactants are of betaine type. Suitable compounds may be selected from alkyl betaines and/or alkylamido betaines. A preferred compound selected from alkyl betaines is lauryl betaine. A preferred compound selected from alkylamido betaines is cocamidopropyl betaine. The disclosure also relates to the salts of the compounds.

Suitable concentration ranges for surfactants are in the range of 0.1% to 10% by weight, calculated to the total weight of the bleaching composition A.

### Optional Compounds—Thickening Polymers

In case the viscosity after mixing with other compositions needs to be further adjusted to prevent dripping, the bleach powder composition may comprise one or more thickening polymers, from the viewpoint of cosmetic safety.

The composition of the present invention comprises one or more thickening polymer(s) selected from non-ionic thickening polymers and/or anionic thickening polymers, and/or their mixtures.

Preferably, the thickening polymers are selected from polymers resulting in an aqueous solution and/or aqueous dispersion at pH between 7 and 12 having a viscosity of at least 1,000 mPa·s measured at a polymer concentration of 1% by weight in water at 25° C., calculated to the total weight of the composition, determined by a Brookfield viscometer, such as at 10 rpm for 1 min, with an appropriate spindle at 25° C.

Suitable non-ionic thickening polymers are cellulose-based polymers. Suitable examples of cellulose-based polymers are methylcellulose, hydroxymethylcellulose, hydroxyethylcellulose, hydroxypropylcellulose, hydroxyethyl-methylcellulose, and alkylated hydroxyl celluloses such as (C₂-C₈)-alkylcelluloses or cetyl hydroxyethylcellulose.

Suitable anionic thickening polymers are selected from naturally-based anionic polymers and/or synthetic anionic polymers.

Suitably, the natural anionic polymer(s) may be selected from xanthan gum, dehydroxanthan gum, hydroxypropylxanthan gum, carboxymethyl cellulose and starch based polymers such as vegetable starch and/or their synthetically modified derivatives such as hydroxypropyl starch phosphate. Equally suitable are alginic acids, sodium alginates, ammonium alginates, calcium alginates, gum arabic, and guar gum.

The preferred thickening polymer for the composition of the present invention are natural anionic polymers, more preferably xanthan gum and/or dehydroxanthan gum, from the viewpoint of their biodegradability and low environmental impact.

Preferably, the total concentration of thickening polymers in the bleaching composition A is 0.1% by weight or more, more preferably 0.25% by weight or more, more preferably 0.5% by weight or more, calculated to the total weight of the bleaching composition A, from the viewpoint of providing sufficient viscosity to the composition.

Preferably, the total concentration of thickening polymers of the bleaching composition A is 15% by weight or less, more preferably 12% by weight or more, further more preferably 10% by weight or less, calculated to the total weight of the composition, from the viewpoint of providing sufficient viscosity to the composition and cost of goods.

For attaining the above-mentioned effects, it is preferred that the total concentration of thickening polymers in the bleaching composition A of the present invention is in the range of 0.1% to 15% by weight, preferably 0.25% to 12% by weight, more preferably in the range of 0.5% to 10% by weight, calculated to the total weight of the bleaching composition A.

### Two-Part Composition

The present invention is also directed to a two-part bleaching composition for keratin fibers, preferably for human keratin fibers, more preferably for human hair, comprising a bleaching composition A as defined above and a second aqueous oxidizing composition B, preferably comprising hydrogen peroxide and having a pH in the range of 1 to 6.

Suitable concentrations of the oxidizing agent(s), preferably hydrogen peroxide, in the aqueous oxidizing composition B is in the range of 1% to 20% by weight, more preferably 2% to 15% by weight, further more preferably 3% to 12% by weight, calculated to the total weight of the second oxidizing composition B.

Suitably, from the viewpoint of stability of the aqueous oxidizing composition B, the pH of the composition is in the range of 1 to 6, more preferably 1.5 to 5, more preferably 2 to 4.5.

The pH may be adjusted with well-known acids such as phosphoric acid.

### Kit-of-Parts

The present invention is also directed to a kit-of-parts for bleaching and dyeing keratin fibers, preferably human keratin fibers, more preferably human hair comprising:
a bleaching composition A as defined above,
a second aqueous oxidizing composition B, preferably comprising hydrogen peroxide and having a pH in the range of 1 to 6,
a third composition comprising one or more dye(s), preferably one or more oxidative hair dye(s) and/or one or more hair direct dye(s), and/or their mixtures.

The second aqueous oxidizing composition may have the same features and characteristics as the second aqueous composition B of the two-part composition.

The third composition comprising one or more dye(s), preferably one or more oxidative hair dye(s) and/or one or more hair direct dye(s), and/or their mixtures may further comprise one or more alkalizing agent(s). Preferably, this composition has a pH in the range of 7 to 12, more preferably in the range of 8 to 11, further more preferably 8.5 to 10.

Suitable alkalizing agents are ammonia and the alkalizing agents as present above for the bleaching composition A. The concentration of alkalizing agents in the dyeing composition may be similar to the bleaching composition A.

### Method for Bleaching

The present invention is also directed to a method for bleaching keratin fibers, preferably human keratin fibers, more preferably human hair, comprising the steps of:
i) mixing a bleaching composition A as defined above with a second oxidizing composition B, preferably comprising hydrogen peroxide and having a pH in the range of 1 to 6, to yield a ready-to-use composition having a pH in the range of 7 to 12,
ii) applying the ready-to-use composition onto keratin fibers and leaving it for a time period of 1 min to 60 min,
iii) rinsing-off the keratin fibers with water and optionally shampooing the keratin fibers.

The bleaching composition A is mixed with the aqueous oxidizing composition B to form a ready-to-use composition. Suitable mixing ratios by weight are 5:1 to 1:5 (bleach powder composition: aqueous oxidizing composition). Customarily, suitable mixing ratios are 1:1, 1:2, and 1:3 by weight (bleaching composition A: aqueous oxidizing composition B).

Suitably, the pH of the ready-to-use composition is in the range of 7 to 12. It is preferred from the viewpoint of accelerated bleaching that the pH of the ready-to-use composition is in the range of 7.5 to 11, more preferably 8.0 to 10.5.

The ready-to-use composition is then applied to keratin fibers and left for a time period of 1 min to 60 min as defined in step ii). Preferred time ranges for step ii) are 5 min to 50 min, more preferred ranges are 10 min to 45 min, from the viewpoint of sufficiently bleaching the keratin fibers.

After that, the ready-to-use composition is rinsed-off from keratin fibers and optionally they are shampooed and optionally blow-dried.

### Method for Dyeing

The present invention is also directed to a method for dyeing of keratin fibers, preferably human keratin fibers, more preferably human hair, comprising the steps of:
iv) providing the bleaching composition A as defined above and mixing it with an aqueous oxidizing composition B, preferably comprising hydrogen peroxide and having a pH in the range of 1 to 6, to yield a ready-to-use composition having a pH in the range of 7 to 12,
v) applying the ready-to-use composition onto keratin fibers and leaving it for a time period of 1 min to 60 min,
vi) rinsing-off the keratin fibers with water and optionally shampooing the keratin fibers,
vii) providing a dyeing composition comprising one or more oxidative dye(s) and/or direct dye(s), and/or their mixtures, and comprising one or more alkalizing agent(s),
viii) mixing the composition of step vii) with an aqueous oxidative composition B to form a ready-to-use dyeing composition having a pH in the range of 7 to 12,
ix) applying the ready-to-use dyeing composition onto keratin fibers and leaving it for a time period of 1 min to 60 min,
x) repeating step vi).

Process steps iv) to vi) are identical to process steps i) to iii) and, therefore, the disclosure of the bleaching process is essentially the same for the bleaching process in combination with the oxidative dyeing process.

The oxidative dyeing composition of step vii) may be the same composition as disclosed for the kit-of-parts.

The following examples are to illustrate the invention, but not to limit it.

### EXAMPLES

TABLE 1
Bleaching composition A

| | |
|---|---|
| Inventive | Comparative |
| example 1 | example 1 |

| | |
|---|---|
| Ingredient | % by weight |

| | | |
|---|---|---|
| Potassium persulfate | 40.0 | 40.0 |
| Ammonium persulfate | 15.0 | 15.0 |
| Sodium metasilicate | 11.0 | 11.0 |
| Mannitol | 0.1 | — |

| | |
|---|---|
| Diatomaceous earth | Ad 100.0 |

| | | |
|---|---|---|
| L* | 40.73 | 36.73 |

### Methods

### Bleaching Method

### For Table 1:

Caucasian hair streaks (21 cm, 2 g per bundle) were purchased from Fischbach+Miller Haar, Laupheim, Germany. The bleaching compositions A of table 1 from above were mixed in a weight ratio of 1:1.4 (bleaching composition A: aqueous oxidizing composition B) with an aqueous oxidizing composition B comprising 6% by weight of hydrogen peroxide to prepare a ready-to-use composition with a pH of 10.0±0.2. 1 g of the ready-to-use compositions were applied onto hair streaks and left for 30 min at room temperature. The hair streaks were then rinsed-off with lukewarm water, shampooed with a shampoo commercially available under the trade name Goldwell Deep Cleansing Shampoo, and blow-dried.

### Lightening Measurements

L* values were measured after bleaching with a Datacolor 45G instrument.

The following examples are within the scope of the present invention.

### Example 2

Bleaching composition A

| | % by weight |
|---|---|
| Hydroxyethylcellulose | 3 |
| Tetrasodium EDTA | 2 |
| Sodium carbonate | 1 |
| Ammonium persulfate | 11 |
| Potassium persulfate | 36 |
| Sodium metasilicate | 10 |
| Mannitol | 0.001 |
| Mineral oil | 8 |
| Diatomaceous Earth | to 100 |

### Example 3

Two-part composition

% by weight

Bleaching composition A

| | |
|---|---|
| Hydroxyethylcellulose | 3 |
| Tetrasodium EDTA | 2 |
| Sodium carbonate | 1 |
| Ammonium persulfate | 11 |
| Potassium persulfate | 36 |
| Sodium metasilicate | 10 |
| Mannitol | 0.5 |
| Mineral oil | 8 |
| Diatomaceous Earth | to 100 |

Aqueous oxidative composition B

| | |
|---|---|
| Cetearyl alcohol | 4.0 |
| Sodium lauryl sulfate | 0.8 |
| Phosphoric acid | q.s. ad pH 2.5 |
| Tetrasodium EDTA | 0.05 |
| Hydrogen peroxide | 6.0 |
| Water | ad 100.0 |

### Example 4

### Kit for Bleaching/Lightening and Oxidative Dyeing

The bleaching composition A and aqueous oxidative composition B of example 3 are combined with the following oxidative dyeing composition:

| | % by weight |
|---|---|
| Cetearyl alcohol | 12 |
| Sodium cetearyl sulfate | 2 |
| Cocamide MEA | 5 |
| Oleic acid | 2 |
| Tetrasodium EDTA | 1 |
| Sodium sulfite | 1 |
| Ammonium hydroxide | 5 |
| Ammonium chloride | 1 |
| Toluene-2,5-Diamine sulfate | 0.75 |
| Resorcinol | 0.10 |
| 4-Chlorresorcinol | 0.25 |
| m-Aminophenol | 0.05 |
| 4-Amino-2-Hydroxytoluene | 0.05 |
| Fragrance | 0.5 |
| Water | ad 100 |

The above composition is adjusted to 9.5 by addition of NaOH/HCl.

1. A bleaching composition A for keratin fibers comprising
a) one or more persalt(s) and/or peroxy salt(s),
b) one or more alkalizing agent(s),
c) one or more non-acetylated sugar alcohol(s), and/or their mixtures,
wherein
a total concentration of compound(s) according to group c) is in a range of 0.0001% to 2.5% by weight, calculated to a total weight of the bleaching composition A,
the bleaching composition A comprises less than 5% by weight of water, calculated to the total weight of the bleaching composition A,
and
one or more compound(s) according to group c) is/are mannitol, xylitol, and/or their mixtures.

2. The bleaching composition A of claim 1, wherein the bleaching composition A comprises compound(s) according to group a) at a total concentration in a range of 10% to 80% by weight, calculated to the total weight of the bleaching composition A.

3. The bleaching composition A of claim 1, wherein the compounds according to group b) are inorganic alkalizing agent(s) selected from metasilicates, carbonates, and/or bicarbonates, and/or their alkali or earth alkali salts, and/or their mixtures.

4. The bleaching composition A of claim 1, wherein the compounds according to group b) are organic alkalizing agent(s) selected from, alkyl- or alkanolamines according to the general structure 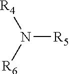
wherein R₄, R₅, and R₆ are same or different H, from C₁ to C₄, C₃ to C₄ unsaturated alkyl, C₃ to C₄ branched alkyl, C₁ to C₄ hydroxyl alkyl, C₃ to C₄ unsaturated hydroxyl alkyl, C₃ to C₄ branched hydroxyl alkyl, with the condition that at least one of R₄, R₅, or R₆ is different from H, and/or their mixtures.

5. The bleaching composition A of to claim 1, wherein the bleaching composition A comprises one or more compound according to group b) at a total concentration in the range of 0.25% to 30% by weight, calculated to the total weight of the bleaching composition A.

6. The bleaching composition A claim 1, wherein the one or more non-acetylated sugar alcohol(s) as compound(s) according to group c) is mannitol.

7. The bleaching composition A of claim 1, wherein a total concentration of compound(s) according to group c) is in a range of 0.0005% to 0.5% by weight, calculated to the total weight of the bleaching composition A.

8. The bleaching composition A claim 1, wherein the bleaching composition A is a bleaching powder composition.

9. The bleaching composition A of claim 1, wherein the bleaching composition A is a bleaching paste composition.

10. The bleaching composition A of claim 1, wherein the bleaching composition A comprises one or more lipophilic compound(s) as compound according to group d).

11. The bleaching composition A of 10, the compound according to group d) is selected from C₁₂ to C₂₂ fatty alcohols, esters of C₃ to C₁₂ alcohols with C₁₂ to C₂₂ fatty acids, C₈ to C₂₂ fatty acids, vegetable oils, and/or silicones, and/or hydrocarbon-based products, and/or their mixtures.

12. The bleaching composition A of claim 10, wherein a concentration of compounds according to group d) is in a range of 1% to 20% by weight, calculated to the total weight of the bleaching composition A.

13. A two-part bleaching composition for keratin fibers, comprising the bleaching composition A of claim 1 to and a second aqueous oxidizing composition B.

14. A kit-of-parts for bleaching and dyeing keratin fibers, comprising:
the bleaching composition A of claim 1,
a second aqueous oxidizing composition B comprising hydrogen peroxide and having a pH range of 1 to 6,
a third composition comprising one or more dye(s).

15. A method for bleaching or dyeing of keratin fibers, comprising the steps of:
i) mixing the bleaching composition A of claim 1 to with a second oxidizing composition B comprising hydrogen peroxide and having a pH range of 1 to 6, to yield a ready-to-use composition having a pH range of 7 to 12,
ii) applying the ready-to-use composition onto keratin fibers and leaving it for a time period of 1 min to 60 min, and
iii) rinsing-off the keratin fibers with water and optionally shampooing the keratin fibers.

16. The method according to claim 15, wherein the method comprises the additional steps of:
iv) providing a dyeing composition comprising one or more oxidative dye(s) and/or direct dye(s), and/or their mixtures, and comprising one or more alkalizing agent(s),
v) mixing the composition of step vii) with an aqueous oxidative composition B to form a ready-to-use dyeing composition having a pH in the range of 7 to 12,
vi) applying the ready-to-use dyeing composition onto keratin fibers and leaving it for a time period of 1 min to 60 min, and
vii) repeating step iii).

17. The bleaching composition A of claim 3, wherein the one or more compounds according to b) is sodium metasilicate.

18. The bleaching composition A of claim 4, wherein the compounds according to b) are selected from monoethanolamine and/or 2-aminomethyl propanol.

19. The bleaching composition A of claim 13, wherein the composition B comprises hydrogen peroxide and has a pH in the range of 1 to 6.

20. The kit of claim 14, wherein the third composition comprises one or more oxidative hair dye(s) and/or one or more hair direct dye(s), and/or their mixtures.
